SEQUENCE LISTING (7 sequences)

SEQ ID NO: 1 - protein, 8 aa, unidentified
WVELHFFN

SEQ ID NO: 2 - protein, 19 aa, unidentified
TFFYGGSRGKRNNFKTEEY

SEQ ID NO: 3 - protein, 6 aa, synthetic construct
PLGLAG

SEQ ID NO: 4 - protein, 11 aa, Human immunodeficiency virus 1
YGRKKRRQRRR

SEQ ID NO: 5 - protein, 44 aa, synthetic construct
TFFYGGSRGKRNNFKTEEYPLGLAGYGRKKRRQRRRWVELHFFN

SEQ ID NO: 6 - protein, 44 aa, synthetic construct; identical to SEQ ID NO: 5
  SITE 1: note = Acetylated residue
  SITE 44: note = Aminated residue

SEQ ID NO: 7 - protein, 4 aa, unidentified
RRRR

(19) United States
(12) Patent Application Publication
(10) Pub. No.: US 2025/0161470 A1
(43) Pub. Date: May 22, 2025

(54) MITOCHONDRIAL INTERVENTION IN NEUROLOGICAL DISORDERS

(71) Applicant: Bimyo GmbH, Monheim (DE)

(72) Inventors: Tienush Rassaf, Essen (DE); Ulrike Hendgen-Cotta, Dinslaken (DE)

(21) Appl. No.: 18/413,733

(22) Filed: Jan. 16, 2024

Related U.S. Application Data
(60) Provisional application No. 63/599,595, filed on Nov. 16, 2023.

(51) Int. Cl. A61K 47/65; A61K 45/06; A61K 47/64

(52) U.S. Cl. CPC A61K 47/65; A61K 45/06; A61K 47/64; A61K 47/6425

(57) ABSTRACT

Approaches and compositions for modulation of mitochondria in the context of neurological disorders are disclosed. Through the disclosure herein, pharmaceuticals are delivered to cells within the brain so as to modulate mitochondrial activity, so as to treat or prevent onset of neurological disorders such as Alzheimer's disease or Parkinson's disease. Delivery is mediated through signals such as polypeptide segments that mediate blood brain barrier passage and neuronal cellular uptake of a pharmaceutical comprising a mitochondrial modulator such as a BNIP3 segment, a structural protein or a transcription factor or transcript stabilizer.

### CROSS-REFERENCE TO RELATED APPLICATIONS

This document claims priority to U.S. Provisional Application Ser. No. 63/599,595, filed Nov. 16, 2023, the contents of which are hereby incorporated by reference in their entirety.

### SEQUENCE LISTING

This application contains polypeptide sequences, listing sequences of residues in order from N- to C-terminal ends. The instant application contains a Sequence Listing which has been submitted electronically in XML format and is hereby incorporated by reference in its entirety. Said XML copy, created on Nov. 20, 2023, is named HNM_003UTL_SL.xml and is 7,673 bytes in size.

### BACKGROUND

Mitochondrial activity is implicated in a broad range of neurological disorders.

For instance, mitochondria are known to play a role in Alzheimer's disease (AD). AD is a neurodegenerative disorder characterized by the accumulation of beta-amyloid plaques and neurofibrillary tangles in the brain. These pathological features are associated with mitochondrial dysfunction, oxidative stress, and impaired energy metabolism, all of which are processes that are regulated by mitochondria.

Similarly, mitochondria are implicated in Parkinson's disease. Parkinson's disease (PD) is a neurodegenerative disorder that affects movement and is characterized by the progressive loss of dopaminergic neurons in the substantia nigra region of the brain. Mitochondrial dysfunction has been implicated in the pathogenesis of PD, as it is responsible for the production of energy in cells and plays a crucial role in maintaining cellular homeostasis.

However, therapeutic delivery to the brain of mitochondrial modulators is problematic. Therapeutics must traverse the blood brain barrier and then be localized within the cytoplasm of brain cells so that they may interact with or be imported into mitochondria.

### SUMMARY

Disclosed herein are compositions comprising a mitochondrial modulator, a blood brain barrier transcytosis mediator, and a neuronal cell plasma membrane transduction mediator. The mediators are often tethered to one another to form a single covalently or noncovalently bound molecule, such as a single polypeptide. The compositions may target defective neuronal mitochondria in an individual suffering from a neurological disorder, such as Parkinson's or Alzheimer's disease.

Similarly, disclosed herein are compositions that modulate neuronal cell mitochondria, in a healthy individual or in an individual suffering from or predicted to develop a neurological disorder such as Parkinson's or Alzheimer's disease. Modulation is direct, through interacting with the mitochondria, or indirect, through triggering changes in neuronal cell expression so as to cause accumulation in the cytoplasm or the mitochondria of mitochondrial mediators, such as proteins that are defective or low or high abundance in the mitochondrial, leading to respiratory chain or other stress in the mitochondria.

Also disclosed here are compositions for use in ameliorating or delaying progression of a neurological disorder, such as Parkinson's or Alzheimer's disease. Such compositions may comprise a mitochondrial modulator, a blood brain barrier transcytosis mediator, and a neuronal cell plasma membrane transduction mediator. The mediators are often tethered to one another to form a single covalently or noncovalently bound molecule, such as a single polypeptide. Similarly, some such compositions modulate neuronal cell mitochondria. Modulation is direct, through interacting with the mitochondria, or indirect, through triggering changes in neuronal cell expression so as to cause accumulation in the cytoplasm or the mitochondria of mitochondrial mediators, such as proteins that are defective or low or high abundance in the mitochondrial, leading to respiratory chain or other stress in the mitochondria.

Disclosed herein are methods of ameliorating a symptom or progression of a neurodegenerative disorder, such as Parkinson's or Alzheimer's disease. Some such methods comprise modulating mitochondrial activity in the brain of an individual at risk of or experiencing a symptom or progression of a neurodegenerative disorder. Similarly, some such methods comprise administering a mitochondrial modulator, a blood brain barrier transcytosis mediator, and a neuronal cell plasma membrane transduction mediator. The mediators are often tethered to one another to form a single covalently or noncovalently bound molecule, such as a single polypeptide.

Also disclosed herein are nucleic acids, such as DNA or RNA, encoding a polypeptide comprising a mitochondrial modulator, a blood brain barrier transcytosis mediator, and a neuronal cell plasma membrane transduction mediator.

Cassettes for neuronal mitochondria delivery are disclosed herein, comprising a blood brain barrier transcytosis mediator and an intracellular localization mediator, often on a common polypeptide backbone and often in combination with a cassette payload such as a mitochondrial modulator. Similarly, nucleic acids encoding such cassettes are disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Disclosed herein are compositions ad methods for treatment, amelioration of the symptoms of, or slowing the progression of neurological disorders such as neurological disorders mediated by mitochondrial perturbations. Through practice of the disclosure herein, on may treat, prevent, ameliorate at least symptom of, or delay the progression of a neurological disorder such as Alzheimer's or Parkinson's disease, such as through mediation, modulation or perturbation of mitochondrial activity in the brain.

Neurological mitochondria modulation is effected through delivery of a mitochondrial modulator to brain cells. Such a delivery is effected in some cases in part through direct administration to the brain of a subject. Alternately, such a delivery comprises administering a composition so as to traverse the blood brain barrier and then to traverse neuronal cell membranes so as to access neuronal mitochondria in the brain. This alternative is substantially less invasive and conducive to routine pharmaceutical administration rather than injection or surgical intervention.

A number of compositions are consistent with the disclosure herein. Compositions generally comprise a mitochondrial modulator, often tethered to a mediator of blood-brain barrier passage and further tethered to a mediator of intracellular delivery. Compositions may further comprise one or more standard of care pharmaceuticals directed to the disorder.

Methods consistent with the disclosure herein variously comprise targeting neurological mitochondrial activity so as to mediate, ameliorate, treat or delay the progression or onset of a neurological disease such as Parkinson's or Alzheimer's disease. Methods variously comprise identifying a patient presenting at least one symptom of a neurological disorder or at risk of developing a neurological disorder such as Parkinson's or Alzheimer's disease, and administering a mitochondrial modulator so as to target neurological mitochondrial activity. Methods variously comprise monitoring the patient for an impact upon the at least one symptom. Administration is single dose or multiple dose, such as at daily, or weekly intervals.

Studies have shown that mitochondrial dysfunction occurs early in the development of AD, before the onset of clinical symptoms. Mitochondrial abnormalities, including decreased mitochondrial number, size, and density, have been observed in the brains of AD patients and animal models of the disease. Additionally, mutations in genes associated with mitochondrial function have been identified in familial forms of AD.

Mitochondria are also involved in the regulation of neuronal signaling and synaptic plasticity, both of which are disrupted in AD. Mitochondrial dysfunction can lead to impaired calcium homeostasis, decreased ATP production, and increased oxidative stress, all of which can contribute to synaptic dysfunction and neuronal cell death.

Overall, while the exact role of mitochondria in the pathogenesis of AD is still not fully understood, there is growing evidence that targeting mitochondrial dysfunction represents a promising approach for developing novel AD therapies.

Mitochondria have been investigated as potential therapeutic targets against Alzheimer's disease (AD). Mitochondrial dysfunction and oxidative stress are known to be involved in the pathogenesis of AD, and as such, targeting mitochondrial function represents a promising approach for developing novel AD therapies.

Several studies have investigated the effects of mitochondria-targeted therapies in preclinical models of AD. For example, some studies have shown that compounds that target mitochondrial function, such as MitoQ and SS31, can improve cognitive function and reduce AD pathology in animal models of the disease.

Other studies have investigated the effects of lifestyle interventions, such as exercise and calorie restriction, which have been shown to improve mitochondrial function and reduce AD pathology in animal models and human studies. Additionally, several clinical trials have investigated the effects of compounds that target mitochondrial function, such as idebenone, a mitochondrial antioxidant, and MitoQ, a mitochondria-targeted antioxidant, in AD patients.

BNIP3 (Bcl-2/adenovirus E1B 19-kDa-interacting protein 3) is a member of the Bcl-2 family of proteins that is involved in regulating apoptosis and autophagy. While BNIP3 has been implicated in neurodegenerative diseases, its exact role in the pathogenesis of PD or AD is still not fully understood.

Some studies have suggested that BNIP3 expression is increased in the brains of PD patients and animal models of the disease, and that it may play a role in mitochondrial dysfunction and oxidative stress, two processes that are known to be involved in the pathogenesis of PD. Additionally, BNIP3 has been shown to interact with other proteins, such as Parkin, PINK1, and Beclin-1, which are known to be involved in the regulation of mitophagy, a process by which damaged or dysfunctional mitochondria are removed from cells.

Similarly, research has indicated that BNIP3 expression was increased in the brains of AD patients and that BNIP3 may play a role in the accumulation of beta-amyloid plaques, a hallmark feature of AD pathology.

There are several mitochondrial targets that have been identified in neurodegenerative diseases, including the following.

Complex I: The electron transport chain (ETC) complex I is the most commonly implicated target in PD. Mutations in mitochondrial DNA (mtDNA) encoding for complex I subunits have been identified in familial forms of PD, and inhibition of complex I activity by toxins such as rotenone and MPTP can cause PD-like symptoms in animal models.

Mitochondrial fission and fusion: Mitochondria undergo frequent fission and fusion events to maintain their shape and function. Dysregulation of these processes has been linked to PD pathogenesis. Several proteins that regulate mitochondrial fission and fusion, such as Drp1, OPA1, and Mfn2, have been shown to be involved in PD.

Mitophagy: Mitophagy is a process by which damaged or dysfunctional mitochondria are selectively removed by autophagy. Impaired mitophagy has been implicated in the accumulation of dysfunctional mitochondria and the development of PD. PINK1 and Parkin are two proteins that play a critical role in the mitophagy pathway and have been linked to PD.

Oxidative stress: Mitochondria are a major source of reactive oxygen species (ROS) in cells. Excessive ROS production can lead to oxidative damage and cell death, and oxidative stress has been implicated in the pathogenesis of PD. Several antioxidants and ROS scavengers have been studied as potential therapeutic targets for PD.

Mitochondrial modulators as disclosed herein may in some cases act to stabilize or ameliorate defects associated with any one or more of these aspects of mitochondrial health in neuronal mitochondria. Similarly, some methods herein serve to trigger expression of proteins that act to stabilize or ameliorate defects associated with any one or more of these aspects of mitochondrial health in neuronal mitochondria.

Neurological mitochondrial modulators. Disclosed herein are compositions related to mitochondrial modulation in neurons or brain cells for the treatment, amelioration, delay or treatment of a neurological disorder such as Parkinson's or Alzheimer's disease.

Many of such compositions comprise at least one of a mitochondrial modulator moiety, a blood brain barrier transcytosis moiety and a neuron plasma membrane transduction mediator, so as to effect delivery of the mitochondrial modulator moiety to neuronal mitochondria. Some such compositions further comprise one or more cleavage linkers so as to facilitate removal of one or more moieties subsequent to localization.

Compositions are often formulated for delivery to the circulatory system of a patient, either through injection, inhalation, consumption or other approach for delivery to the circulatory system of a patient. Alternate delivery approaches not involving the circulatory system are also consistent with the disclosure herein.

Often, the one or more of at least one of a mitochondrial modulator moiety, a blood brain barrier transcytosis moiety and a neuron plasma membrane transduction mediator are tethered as a single molecule, such as a molecule sharing a common polypeptide backbone. The common polypeptide backbone may in some cases further comprises a polypeptide cleavage site, so as to facilitate removal of one or more constituents subsequent to localization. Other tethers, such as Peg linkers or other covalent or noncovalent linkers are also contemplated. Consistent with a polypeptide backbone but also with other covalent or noncovalent linkers, one or more of at least one of a mitochondrial modulator moiety, a blood brain barrier transcytosis moiety and a neuron plasma membrane transduction mediator are polypeptide segments.

The mitochondrial modulator moiety is often a segment of a polypeptide mitochondrial modulator such as BNIP3. The segment is in some cases an N-terminal BNIP3 segment, such as a segment comprising residues 1-20 of BNIP3, or a segment exhibiting 95%, 90%, 85%, 80%, 75%, 70%, 65%, 60%, 55%, 505 or less than 50% identity to the BNIP3 N-terminal 20 residues. The segment in some cases comprises or consists of the residues WVELHFFN (SEQ ID NO: 1), or has a portion exhibiting at least 87.5%, 75%, 62.5%, 50%, 37.5%, or less than 37.5% identity to the residues WVELHFFN (SEQ ID NO: 1), or having a portion exhibiting at least 87.5%, 75%, 62.5%, 505, 37.5%, or less than 37.5% similarity to the residues WVELHFFN (SEQ ID NO: 1).

The blood brain barrier transcytosis mediator is also in some cases a polypeptide segment. In some cases the blood brain barrier is traversed via receptor mediated transcytosis, such as through a receptor or a ligand tethered to the mitochondrial modulator. Some exemplary ligands bind to the receptor lipoprotein receptor-related protein 1 (LRP1), such as aprotinin or angiopeptide, or a polypeptide exhibiting at least 95%, 90%, 85%, 80%, or 75% identity to a polypeptide such as aprotinin or angiopeptide. See, e.g., Habib and Singh (2022) “Angiopep-2-Modified Nanoparticles for Brain-Directed Delivery of Therapeutics: A Review” Polymers (Basel). 2022 February; 14 (4): 712. Published online 2022 Feb. 12. doi: 10.3390/polym14040712, which is hereby incorporated by reference in its entirety, as well s references cited therein.

An exemplary transcytosis mediator is the polypeptide Angiopep-2, a 19 residue polypeptide that binds to LRP1 to mediate blood brain barrier transcytosis. Angiopep-2 has a sequence TFFYGGSRGKRNNFKTEEY (SEQ ID NO: 2); however, polypeptides differing from the sequence of angiopep-2 by 1, 2, 3, 4, 5, 6, 7, 8, 9, 10 or more than 10 residues, in some cases while serving as a ligand to LRP1 or an alternate receptor are also contemplated.

The transcytosis mediator is in some cases linked to the remainder of the molecule via a cleavage moiety, such as a polypeptide cleavage moiety. An exemplary cleavage moiety has the sequence PLGLAG (SEQ ID NO: 3) and is the substrate for cleavage by matrix metalloproteases MMP2 and MMP9. See, for example, Cieplak and Strongin (2017) “Matrix metalloproteinases—from the cleavage data to the prediction tools and beyond.” Biochim Biophys Acta. 2017 November; 1864 (11 Pt A): 1952-1963. A broad range of polypeptide cleavage sites are known in the art, and other cleavage linkers are contemplated herein. Cleavage moieties in some cases act to release adjacent localization signal motifs subsequent to localization. In this case, Angiopep-2 is released subsequent to translocalization of the polypeptide therapeutic across the blood brain barrier. Release of the blood brain barrier translocalization signal in some cases facilitates activates or facilitates activity of the cellular localization motif such as TAT.

The neuronal plasma membrane transduction mediator effects entry of the mitochondrial modulator such as a BNIP3 segment into the neurons so s to access the neuronal mitochondria. Some such plasma membrane transduction mediators are polypeptides spanning all or a portion of a viral transduction domain such as an HIV-1 transduction domain tat peptide. An exemplary transduction domain spans, for example, HIV-1 TAT protein transduction domain48-59 of HIV, or spans a polypeptide having sequence YGRKKRRQRRR (SEQ ID NO: 4), or differs not at all or by no more than 1, 2, 3, 4, 5, or more than 5 residues from HIV-1 TAT protein transduction domain48-59 of HIV, and in some cases exhibits plasma membrane transduction mediation activity. See, e.g., Humaidan et al. (2022) “The Cell-Penetrating Peptide Tat Facilitates Effective Internalization of PSD-95 Inhibitors Into Blood-Brain Barrier Endothelial Cells but less Efficient Permeation Across the Blood-Brain Barrier In Vitro and In Vivo” Frontiers in Drug Delivery April 2022|Volume 2|Article 854703.

Accordingly, some compositions comprise a covalently bound modular molecule such as a polypeptide having at portions that align with at least 80% identity to a mitochondrial modulator having a sequence WVELHFFN (SEQ ID NO: 1), or a nonidentical functional equivalent, at least 80% identity to a blood brain barrier transcytosis mediator having a sequence TFFYGGSRGKRNNFKTEEY (SEQ ID NO: 2), or a nonidentical functional equivalent, and at least 80% identity to a plasma membrane transduction mediator having a sequence YGRKKRRQRRR (SEQ ID NO: 4), or a nonidentical functional equivalent. The order of these three segments within the molecule is not specified in all cases, such that a mitochondrial modulator, a blood brain barrier transcytosis mediator, and a plasma membrane transduction mediator all may variously appear, first, second or third as measured from the n-terminus of the polypeptide or of the first segment of the modular molecule. The segments are linked directly or in some cases are tethered to one another by one or more linkers, such as two linkers, for example polypeptide linkers which may in some cases share a common polypeptide backbone with the segments. In some cases the percent identity is greater than 80%, such as 85%, 90%, 95%, 98%, 99%, or 100% for one or more of the segments. The linker may be a cleavable linker, such as PLGLAG (SEQ ID NO: 3), a polypeptide 80% identical to PLGLAG (SEQ ID NO: 3), or a nonidentical functional equivalent.

Some exemplary embodiments comprise or consist of the polypeptide sequence TFFYGGSRGKRNNFKTEEYPLGLAGYGRKKRRQRRRWVELHFFN (SEQ ID NO: 5), comprising an Anigopep-2 motif, an MMP2/9 cleavage domain, an HIV TAT localization motif, and a BNIP3 optimized region. Alternately, some exemplary embodiments differ from the sequence listed such that they exhibit at least 99%, 97%, 95%, 93%, 91%, 90%, 85%, 80% or less than 80% identity or similarity to the sequence listed. Percent identity or similarity may be determined using local BLAST alignment tools available at the US National Center for Biotechnology Information website, or may be determined using other alignment tools commonly employed in the art.

Some exemplary embodiments comprise Anigopep-2 motif or other blood brain barrier translocalization motif, an MMP2/9 or other cleavage domain, an HIV TAT or other cellular localization motif, and a BNIP3 optimized region or other mitochondrial modulator, adjacent or separated by intervening linker sequence on a common polypeptide backbone.

The polypeptide may further comprise a portion encoding a moiety suitable for purification of the polypeptide, such as a His-tag or an epitope suitable for purification, and may further comprise a polypeptide suitable for cleavage of the purification moiety subsequent to purification. These features are particularly beneficial when polypeptides are synthesized via translation of a transcribed RNA molecule encoding the product.

Alternately, in some exemplary embodiments the polypeptide is synthesized chemically. Such an approach facilitates pure compositions of polypeptides. Some such approaches involve N-alpha-Fmoc protected amino acids, TFA mediated cleavage, HPLC purification and ion exchange. A number of chemical polypeptide synthesis approaches are known in the art and consistent with synthesis of the polypeptides herein.

Some polypeptides herein are synthesized voia translation, and the resultant products comprise an N-terminal Methionine and A Carboxy C-terminus.

Alternately, in particular for chemically synthesized polypeptides, the resultant molecule comprises an N-terminal, C-terminal or both N-terminl and C-terminal cap, such as an Acetyl N-terminal cap and an amino C-terminal cap. An exemplary polypeptide arising from chemical syntesis is thus Ac-TFFYGGSRGKRNNFKTEEYPLGLAGYGRKKRRQRRRWVELHFFN-NH₂ (SEQ ID NO: 6). In these molecules the “Ac-” represents an Acetyl cap and the “—NH₂” represents an amino C-terminal cap, rather than representing amino acid residues in the polypeptide.

Some compositions further comprise a buffer or carrier compatible with ingestion, injection, inhalation, absorption across a membrane or skin layer, or other approach for introduction of the active moiety of the composition to the blood brain barrier.

Some compositions further comprise a second active ingredient or are formulated for administration concurrently or in combination with a second active ingredient. Compositions directed to Parkinson's disease, for example, comprise or are formulated for administration concurrently or in combination with a dopamine precursor such as levodopa; carbidopa; a Catechol-O-methyl transferase (COMT) inhibitor such as one selected from the list consisting of Entacapone, tolcapone, and Opicapone; amantadine; an adenosine A2A antagonist; istradefylline; an Anticholinergic medication, such as one selected from the list consisting of Benztropine and Trihexylphenidyl HCl; a MAO-B inhibitor such as one selected from the list consisting of Selegiline, Selegiline HCl, Rasagiline, and Safinamide; or other active ingredient associated with Parkinson's treatment.

Compositions directed to Alzheimer's disease, for example, comprise or are formulated for administration concurrently or in combination with a beta-amyloid clearance therapeutic such as one selected from the list consisting of aducanumab or lecanemab; a mitochondrial mediator such as MitoQ and SS31, or idebenone; an acetylcholine mediator; or a cholinesterase inhibitor such as one selected from the list consisting of Galantamine (Razadyne®), Rivastigmine (Exelon®), and Donepezil (Aricept®). Compositions are in some cases administered in combination with caloric restriction regimens.

Alternative mitochondrial modulators are also contemplated herein. For example, some mitochondrial modulators act on mitochondria indirectly, such as by eliciting expression of nuclear encoded mitochondrially localized stabilizing proteins, such as Electron transport chain complex 1 constituents or stabilizers, mitochondrial fission or fusion modulators such as Drp1, OPA1, and Mfn2; mitophagy modulators such as Beclin-1, PINK1 and Parkin; or Reactive Oxygen species scavengers such as protein antioxidants or superoxide dismutase or other ROS scavengers. Upon expression of such proteins and in some cases localization of such proteins to the mitochondria, one may effect mitochondrial stabilization, mitochondrial function stabilization, or clearance of dysfunctional representatives in a cellular mitochondrial population and their replacement by progeny of healthy mitochondria in the cellular mitochondrial population.

Alternative mitochondrial modulators may comprise a blood brain barrier transcytosis mediator, such as one having at least 80% identity to a polypeptide having a sequence TFFYGGSRGKRNNFKTEEY (SEQ ID NO: 2), and an intracellular localization mediator, such as one having at least 80% identity to a plasma membrane transduction mediator having a sequence YGRKKRRQRRR (SEQ ID NO: 4). The motifs are optionally linked by a cleavable linker, such as an exemplary cleavage moiety having the sequence PLGLAG (SEQ ID NO: 3) and that acts as the substrate for cleavage by matrix metalloproteases MMP2 and MMP9. Alternately or in addition, some mitochondrial modulators comprise a nuclear localization signal such as an RRRR motif (SEQ ID NO: 7) or other nuclear localization motif known in the art. The modulator itself may comprise a portion or all of a transcription factor that directs expression of a mitochondrial protein such as a protein listed above, or a protein that modulates such a transcription factor or modulates such protein directly. Similarly, transcription factors or stability factors modulating expression of mitochondrially encoded mitochondrial components are also contemplated and consistent with the disclosure herein. Transcription factors may be portions or full length proteins fused to localization signals disclosed herein, or may comprise synthetic transcription factors such as CRISPR, Zinc finger, beta-helix-loop-helix or other artificially designed nucleic acid binding transcription factors.

Common to may of these compositions are polypeptide segments including a segment that mediates blood brain barrier transcytosis, and an intracellular localization mediator directing uptake and in some cases cytoplasmic, mitochondrial or nuclear localization in cells within the neuronal cells. Nuclear localization segments may be beneficial for directing transcription factors to the nucleus, while mitochondrial localization signals, such as N-terminal mitochondrial localization leader segments known in the art, may direct structural or transcription factor or transcript stabilization factors to the mitochondria to modulate mitochondrial expression. The segments may share a common polypeptide backbone or may be otherwise tethered to one another.

In light of the components disclosed above and know in the art, cassettes for neuronal mitochondria delivery are disclosed herein. Some such cassettes comprise a blood brain barrier transcytosis mediator such as those disclosed herein, and an intracellular localization mediaton alone, substituted by or in combination with other localization component. Cassettes are often on a common polypeptide backbone, that is, polypeptides. Cassettes are configured to deliver a payload to neuronal cells so as to modulate neuronal mitochondria. Cassette payloads are often polypeptide segments sharing common polypeptide backbones with the remainder of the cassette, though alternatives are also contemplated. Exemplary cassette payloads include BNIP3 segments, transcription factor segments, or transcript stabilization segments, and may be configured for mitochondrial, cytoplasmic or nuclear localization. Similarly, nucleic acids encoding such polypeptide or other cassettes are disclosed herein.

Methods of neurological disorder treatment. Disclosed herein are methods of neurological treatment, symptom amelioration, prevention or delay of progression or a neurological disorder such as Parkinson's or Alzheimer's disease, comprising modulating neurological mitochondrial activity. Through practice of the methods herein, mitochondrial activity is modulated in the brain of an individual having or at risk of having a neurological disorder such as Parkinson's or Alzheimer's disease, so as to treat, ameliorate a symptom of, or delay onset or progression of a neurological disorder.

Some such methods comprise stabilizing neurological mitochondria so as to prevent neurological decline at the cellular level.

Stabilizing is effected through a broad range of approaches. In some cases a mitochondrial modulating agent or moiety is delivered to neurological cell mitochondria, so as to effect mitochondrial modulation within the brain. Such approaches require that a mitochondrial modulator or stabilizer be delivered across the blood brain barrier and then be taken up intracellularly, so as to deliver a mitochondrial modulator to brain mitochondria.

Pursuant to practice of some approaches to the method, mitochondria in the brain of a subject or patient are stabilized via delivering a mitochondrial modulator across the blood brain barrier and to intracellular regions of a subject's brain. The modulator may act on mitochondria directly, or may trigger changes in gene expression so as to modulate mitochondria in neuronal cells.

A subset of the methods herein comprise administration of a neurological mitochondrial modulator or other mitochondrial modulating moiety so as to stabilized neurological mitochondria. Pursuant to these methods, one or more additional active agents may administered, directed to the disorder being targeted. Such additional agents may be standard of care compositions for a target disorder or may be intended to support the activity of a neurological mitochondrial modulator.

For example, for a method of addressing Parkinson's, one may administer concurrently or as part of a common treatment regimen a dopamine precursor such as levodopa; carbidopa; a Catechol-O-methyl transferase (COMT) inhibitor such as one selected from the list consisting of Entacapone, tolcapone, and Opicapone; amantadine; an adenosine A2A antagonist; istradefylline; an Anticholinergic medication, such as one selected from the list consisting of Benztropine and Trihexylphenidyl HCl; a MAO-B inhibitor such as one selected from the list consisting of Selegiline, Selegiline HCl, Rasagiline, and Safinamide; or other active ingredient associated with Parkinson's disease treatment.

For a method of addressing Alzheimer's disease, for example, one may administer concurrently or as part of a common treatment regimen a beta-amyloid clearance therapeutic such as one selected from the list consisting of aducanumab or lecanemab; a mitochondrial mediator such as mitoQ, SS31, or idebenone; an acetylcholine mediator; or a cholinesterase inhibitor such as one selected from the list consisting of Galantamine (Razadyne®), Rivastigmine (Exelon®), and Donepezil (Aricept®). Compositions are in some cases administered in combination with caloric restriction regimens.

A treatment regimen may comprise a single dose administration, or multiple administrations, for example daily, every few days, weekly, or 2, 3, 4, 5, 6, or more than 6 administrations per day over a single day or multiple days. Administration may be performed over a single definite duration, or may be indefinite or ongoing. Administration duration may be informed by an ongoing monitoring regimen, such that administration timing or dosage is modulated in light of patent symptom response to the treatment regimen.

Additive agents may be administered in a common formulation or independently as part of a common treatment regimen.

A patient may be selected for treatment based upon presentation of symptoms of a neurological disorder, based upon knowledge of a familiar predisposition for a neurological disorder, based upon a history of brain trauma predictive of neurological disease, based upon identification of an allele or alleles indicative or predictive of a neurological disorder, or basec upon other criteria.

The subject or patient may exhibit one or more symptoms of a neurological disorder such as Parkinson's or Alzheimer's disease, or may be at risk of development of a neurological disorder such as one mentioned herein or known in the art. A symptom may be measured prior to administration of a mitochondrial modulator, and may be measured during, subsequent to or both during and subsequent to a treatment regimen comprising administration of a mitochondrial modulator composition such as those disclosed herein.

A patient is administered a composition such as the neurological mitochondrial modulator compositions disclosed elsewhere herein. Administration is variously oral, through injection, across the skin or a membrane, through inhalation or other administration approach known in the art. Administration is effected so as to allow the neurological mitochondrial modulator composition, or a neurological mitochondrial modulator composition constituent, to arrive at and traverse the blood brain barrier. That is, administration routes are selected so as to allow the neurological mitochondrial modulator composition or a constituent thereof to circulate and be presented to the blood brain barrier at sufficient concentrations to effect mitochondrial modulation upon delivery to the neurological mitochondria.

Pursuant to practice of a method or administration of a composition herein, in some cases one observes an amelioration of at least one symptom of at least 10%, 20%, 30%, 40%, 50%, 60%, 70%, 80%, 90% or more than 90%. Representative symptoms include, for example, frequency or severity of tremors, or muscular control for Parkinson's patients, amyloid accumulation as measured, for example, by MRI scan, performance on a cognition or memory test, facial recognition, or muscular control for Alzheimer's disease. Similarly, in some cases one sees, a delay in disease progression of at least 10%, 20%, 30%, 40%, 50%, 60%, 70%, 80%, 90% or more than 90% relative to an expected rate of disease progression, as determined by assay performance or benchmark condition status. Similarly, in some cases one sees a delay in disease onset for at least 1 month, 2 months, 60 months, 1 year, 5 years or longer.

Compositions for manufacture of medicaments. Disclosed herein are nucleic acids encoding polypeptides having portions that align with at least 75% identity to a mitochondrial modulator having a sequence WVELHFFN (SEQ ID NO: 1), at least 80% identity to a blood brain barrier transcytosis mediator having a sequence TFFYGGSRGKRNNFKTEEY (SEQ ID NO: 2), and at least 80% identity to a plasma membrane transduction mediator having a sequence YGRKKRRQRRR (SEQ ID NO: 4). The order of the regions encoding these three segments within the nucleic acid molecule is not specified in all cases, such that a mitochondrial modulator, a blood brain barrier transcytosis mediator, and a plasma membrane transduction mediator all may variously appear, first, second or third as measured from the n-terminus of the encoded polypeptide. In some cases the percent identity is greater than 80%, such as 85%, 90%, 95%, 98%, 99%, or 100% for one or more of the segments. The nucleic acids are in some cases DNA molecules, and may further comprise a promoter region so as to drive transcription of the portion of the nucleic acids encoding the polypeptides. The nucleic acids are in some cases RNA molecules, and may further comprise a 5′ methylctytosie cap or a 3′ polyadeylated tail or both a cap and tail. The nucleic acids may further comprise a portion encoding a moiety suitable for purification of the encoded polypeptide, such as a His-tag or an epitope suitable for purification, and may further comprise a region encoding a polypeptide suitable for cleavage of the purification moiety subsequent to purification.

The nucleic acids may be harbored on an expression vector for in vitro expression, or within an in vivo expression system such as a eubacterial, archaeal, or eukaryotic cell based or viral expression system.

### Definitions

As used herein, percent identity represents the percent of residues in a segment of a first polypeptide that match in order and chemical composition the residues of a second polypeptide over a similar length.

As used herein, percent similarity represents the percent of residues in a segment of a first polypeptide that match in order and in general chemical properties the residues of a second polypeptide over a similar length.

As used herein, the term “about” in reference to a number refers to a range spanning 10% below that number to 10% above that number, while in reference to a range refers to an extended range spanning from 10% below the lower limit to 10% above the upper limit stated for the range.

We claim:

1. A composition comprising a blood brain barrier transcytosis mediator, a neuronal plasma membrane transduction mediator and a mitochondrial modulator tethered as a common molecule, wherein the blood brain barrier transcytosis mediator and the neuronal plasma membrane transduction mediator are separated by a metalloprotease cleavage site polypeptide, wherein the mitochondrial modulator comprises a polypeptide at least 85% identical to the octapeptide WVELHFFN (SEQ ID NO: 1), wherein the blood brain barrier transcytosis mediator comprises a polypeptide at least 85% identical to TFFYGGSRGKRNNFKTEEY (SEQ ID NO: 2), wherein the cleavage motif comprises a polypeptide differing by at most one residue from the polypeptide PLGLAG (SEQ ID NO: 3), wherein the neuronal plasma membrane transduction mediator comprises a polypeptide at least 85% identical to the polypeptide YGRKKRRORRR (SEQ ID NO: 4), and wherein the mitochondrial modulator, the neuronal plasma membrane transduction mediator, the cleavage motif and the neuronal plasma membrane transduction mediator share a common polypeptide backbone.

2. The composition of claim 1, formulated for delivery to a patient.

3. The composition of claim 1, wherein the mitochondrial modulator comprises the polypeptide WVELHFFN (SEQ ID NO: 1), wherein the blood brain barrier transcytosis mediator comprises the polypeptide TFFYGGSRGKRNNFKTEEY (SEQ ID NO: 2), wherein the cleavage motif comprises the polypeptide PLGLAG (SEQ ID NO: 3), wherein the neuronal plasma membrane transduction mediator comprises the polypeptide YGRKKRRORRR (SEQ ID NO: 4), and wherein the mitochondrial modulator, the neuronal plasma membrane transduction mediator, the cleavage motif and the neuronal plasma membrane transduction mediator share a common polypeptide backbone.

4.-13. (canceled)

14. The composition of claim 1, wherein the neuronal plasma membrane transduction mediator comprises a polypeptide having 100% identity to YGRKKRRQRRR (SEQ ID NO: 4).

15. (canceled)

16. The composition of claim 1, wherein the polypeptide of the common polypeptide backbone comprises a polypeptide having a sequence at least 85% identical to the sequence TFFYGGSRGKRNNFKTEEYPLGLAGYGRKKRRQRRRWVELHFFN (SEQ ID NO: 5), and wherein the polypeptide exhibits mitochondrial modulatory activity.

17. The composition of claim 16, wherein the polypeptide of the common polypeptide backbone comprises a polypeptide having the sequence TFFYGGSRGKRNNFKTEEYPLGLAGYGRKKRRQRRRWVELHFFN (SEQ ID NO: 5).

18. The composition of claim 17, formulated for delivery to a patient.

19. The composition of claim 18, further comprising a dopamine precursor.

20. The composition of claim 18, further comprising at least one medicament selected from the list consisting of a beta-amyloid clearance therapeutic, aducanumab, lecanemab, a mitochondrial mediator, and a cholinesterase inhibitor.

21. A composition comprising a polypeptide having a sequence at least 85% identical to the sequence TFFYGGSRGKRNNFKTEEYPLGLAGYGRKKRRQRRRWVELHFFN (SEQ ID NO: 5).

22. The composition of claim 21, wherein the polypeptide has a sequence at least 90% identical to the sequence TFFYGGSRGKRNNFKTEEYPLGLAGYGRKKRRQRRRWVELHFFN (SEQ ID NO: 5).

23. The composition of claim 21, wherein the polypeptide has a sequence at least 95% identical to the sequence TFFYGGSRGKRNNFKTEEYPLGLAGYGRKKRRQRRRWVELHFEN (SEQ ID NO: 5).

24. The composition of claim 21, wherein the polypeptide has the sequence TFFYGGSRGKRNNFKTEEYPLGLAGYGRKKRRQRRRWVELHFFN (SEQ ID NO: 5).

25. The composition of claim 24, wherein the polypeptide has the structure Ac-TFFYGGSRGKRNNFKTEEYPLGLAGYGRKKRRQRRRWVELHFFN-NH₂ (SEQ ID NO: 6), wherein “Ac-” represents an Acetyl cap and “—NH₂” represents an amino C-terminal cap.

26. The composition of claim 21, formulated for delivery to a patient.

27. The composition of claim 21, further comprising a dopamine precursor.
